(19) United States
(12) Patent Application Publication
(10) Pub. No.: US 2026/0151254 A1
(43) Pub. Date: Jun. 4, 2026

(54) EXTERNAL URINE RECEIVER

(71) Applicant: Medline Industries, LP, Northfield, IL (US)

(72) Inventor: Michael Gerskovich, Arlington Heights, IL (US)

(21) Appl. No.: 18/967,463

(22) Filed: Dec. 3, 2024

(51) Int. Cl. A61F 5/443; A61F 5/453

(52) U.S. Cl. CPC A61F 5/443; A61F 5/453

(57) ABSTRACT

A urine receiver includes a liquid-impermeable pouch having (a) an opening sized for placement adjacent a female urethra or (b) a penis-receiving opening. The opening communicates with an interior of the pouch. The pouch has a first end and a second end and a first side and a second side. The receiver includes a body-connecting region having adhesive disposed thereon for connection of the receiver to the body of a patient, and a fluid discharge tube proximal the first end of the pouch. The tube communicates with the exterior of the pouch and fluidically communicates with an interior conduit. The conduit may terminate in a manifold or may be a loop conduit with urine-receiving perforations.

### FIELD

The disclosure is in the field of incontinence management.

### BACKGROUND

It is necessary to manage urine output for patients that are comatose, under sedation, or otherwise unable to manage their own urine output. The art has provided numerous devices, in particular, indwelling catheters for management of urine. Indwelling catheters are prone to infection and the development of CAUTI—Catheter Associated Urinary Tract Infection—a serious form of infection that can lead to sepsis and severe adverse consequences for the patient.

In recognition of this, more recently the art has provided a number of urine receivers, sometimes called “catheters,” that are designed to be positioned externally and not as indwelling devices. These devices are capable of storing a small amount of discharged urine but principally are intended as receivers to direct the urine to a discharge channel or tube and into a separate receptacle. In use, such devices are placed adjacent the male or female urethra and connected indirectly to a suction source such as a suction pump or house suction, with the separate receptacle intermediating the devices and their respective suction sources.

Now disclosed are urinary receivers having internal tube structures that are designed for rapid urine removal from the receivers via a discharge tube. The receiver may include an internal conduit that terminates in a manifold, an internal loop conduit, or a perforated conduit, and in either case may include one or more internal support struts and spacers.

### BRIEF DESCRIPTION OF THE DRAWINGS

FIG. 1 is a schematic representation of a urine discharge system;

FIG. 2 is a schematic representation of the urine discharge system of FIG. 1, shown in use with a male patient.

FIG. 3 is a front elevation of a prior art urine receiver;

FIG. 4 is a rear elevation of the prior art urine receiver of FIG. 3;

FIG. 5 is a front elevation depicting the internal tubing in the prior art urine receiver shown in FIGS. 3 and 4;

FIG. 6 is a front elevation showing the internal tubing and support strut of a first embodiment of the urine receiver described herein;

FIG. 7 is a representational front elevation of a first embodiment of the urine receiver described herein having internal tubing and a support strut as shown in FIG. 6;

FIG. 8 is a front elevation depicting alternative internal tubing in a urine receiver, including an alternative manifold;

FIG. 9 is a front elevation of the internal tubing loop of another embodiment of the urine receiver described herein;

FIG. 10 is a relatively enlarged perspective view illustrating a perforated section of the tubing shown in FIG. 10.

FIG. 11 is a representation of an embodiment of the urine receiver described herein having an internal tubing loop as shown in FIG. 10;

FIG. 12 is a front elevation, partially cut away, of another embodiment, illustrating a urine receiver with two support struts;

FIGS. 13 and 14 are perspective views of alternative embodiments of urine receivers as described herein, the internal tubing thereof shown in hidden lines.

FIGS. 15 and 16 are front elevations of two further embodiments of urine receivers.

FIGS. 17 and 18 are front elevations depicting yet additional embodiments of urine receivers.

FIG. 19 is a representation of a urine receiver that includes struts, a perforated tube, and a manifold.

FIG. 20 is a representation of a further alternative urine receiver.

The FIGURES are in some parts representational. Terms of orientation are with internal reference to the FIGURES and are not intended to limit the orientation or use of the device.

### DESCRIPTION

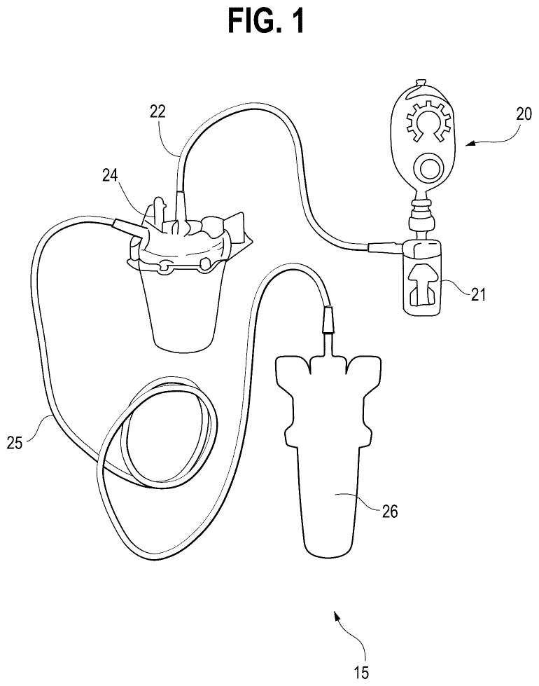

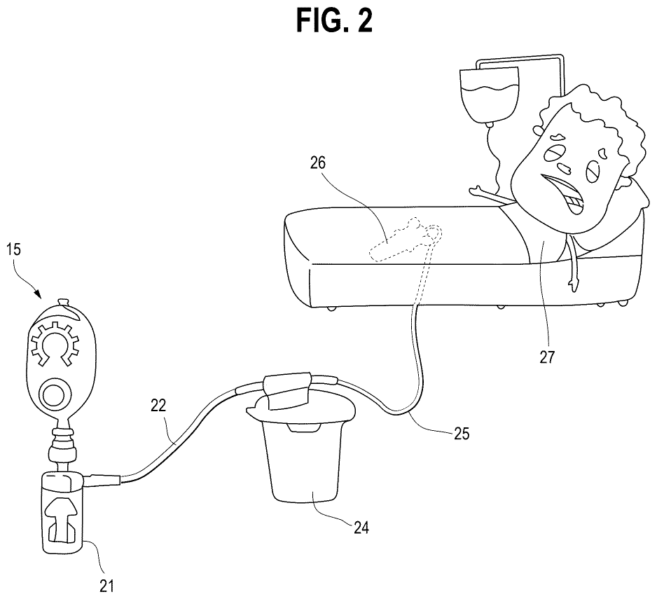

The urine discharge system 15 shown in FIG. 1 includes a suction source 20, which may be a house vacuum system (not shown) in a hospital setting or a separate suction pump 21 as shown. The suction pump 21 is connected via first tubing 22 to a urine canister 24. The urine canister 24 is connected via second tubing 25 to a urine receiver 26. In use, as shown in FIG. 2, the urine receiver 26 is placed proximal the urethra of male patient 27 in a manner effective to collect urine discharged by the patient 27. As the patent 27 discharges urine, pressure generated by the pump 21 creates suction in the canister 24, which draws urine from the urine receiver 26 through second tubing 25 and into the urine canister 24. The urine canister 24 may be graduated to assist in measuring urine output. Via monitoring and periodic emptying of the urine canister 24, urine can be prevented from passing though first tubing 22 into the pressure pump 21 or into the house vacuum system. In some embodiments, a computerized or other monitoring system may be employed. For example, the canister may be equipped with a level sensor that triggers an alarm once the amount of urine has reached a recommended removal level, or may be equipped with a sensor that evaluates a condition of the urine and a processor that generates an alarm or a report based on the condition evaluated

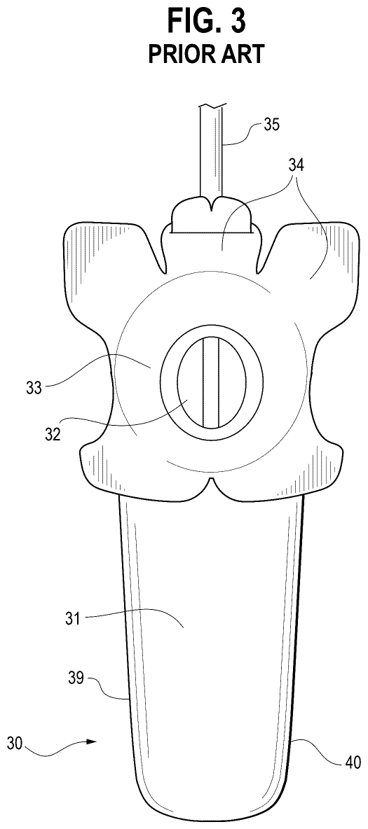

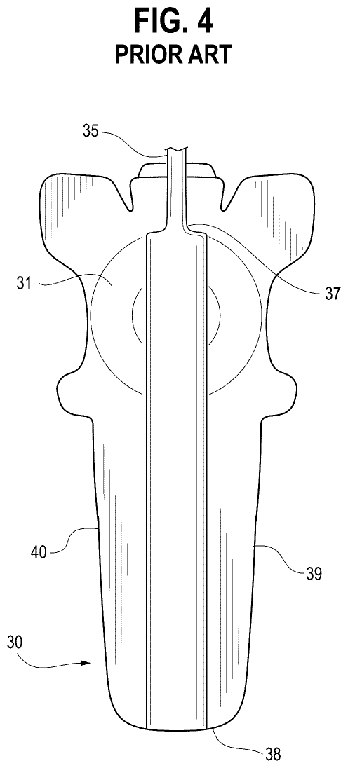

As seen in FIGS. 3 and 4, the prior art urine receiver 30 includes a fluid-impermeable pouch 31 having a penis-receiving opening 32 that leads into the interior of the pouch 31. The pouch 31 may be made from any suitable flexible plastic material, and may have nonwoven material (such as spunbond-meltblown-spunbond material) disposed in the interior surface for patient comfort and to impart opacity for purposes of patient dignity. The pouch may alternatively or in addition thereto be formed from an opaque or translucent plastic material. The pouch has a generally gusseted structure to allow expansion to accommodate the patient's penis and some discharged urine.

The receiver 30 includes a body contact region 33 having adhesive and removable backing material 34 to allow adhesive securement to the patient. In use, a caregiver will remove the adhesive backing material 34 and secure the receiver 30 to the patient, placing the patient's penis into the penis-receiving opening 32.

A discharge tube 35 communicates with the interior of the pouch 31 and with internal tubing as discussed further below. The discharge tube 35 is connected or connectable to second tubing 25 of a urine discharge system as shown in FIG. 1. The pouch 31 has a first end 37 (FIG. 4 only) generally intended to be the proximal or upper end of the pouch, and a second end 38 generally intended to be the lower or distal end of the pouch 31. The pouch 31 also has a first side 39 and a second side 40. More details about this device can be found in U.S. Publication No. 2023/0190510, incorporated herein by reference in its entirety.

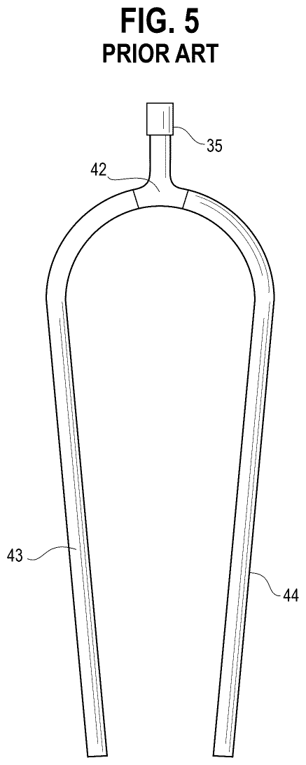

As shown in FIG. 5, the discharge tube 35 is connected via connector 42 to a first internal tube 43 and to a second internal tube 44. Connector 42 can be a “tee”/“T” connector or “wye”/“Y” connector or any other type of connector which allows for a fluid connection between discharge tube 35, first internal tube 43 and second internal tube 44. The internal tubes 43, 44 flank the penis-receiving opening 32 and each extend generally from the first end 37 to the second end 38 of the pouch, where tube 43 extends generally along the first side 39 of the pouch and tube 44 extends generally along the second side 40 of the pouch (pouch not shown in FIG. 5). The internal tubes 43, 44 are designed to allow urine to flow from the interior of the pouch and outwards through discharge tube 35.” Also, the internal tubes 43, 44 can have a degree of stiffness which allows the tubes to impart some structural support to the pouch 31.

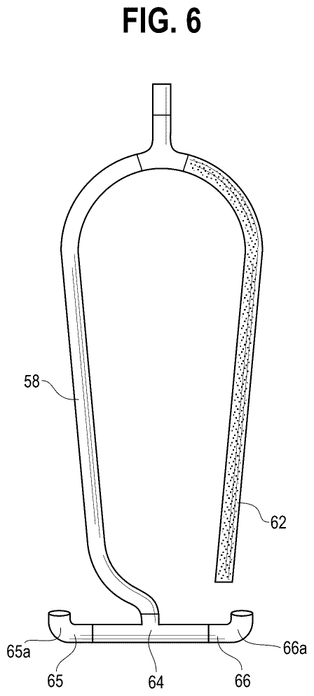

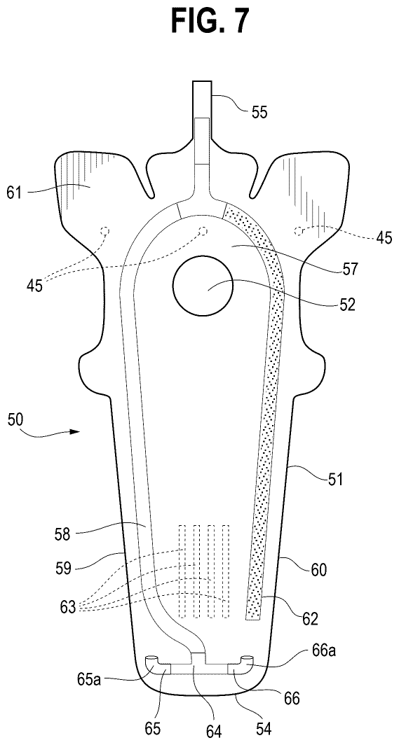

As seen in FIGS. 6 and 7, a first embodiment of the urine receiver 50 (FIG. 7 only) disclosed herein has a liquid-impermeable pouch 51 having a penis-receiving opening 52 that communicates with an interior of the pouch 51. The pouch 51 has a first end 57 and a second end 54 and a first side 59 and a second side 60. The urine receiver 50 includes a body-connecting region 61 having adhesive disposed thereon for connection of the receiver 50 to the body of a patient, as discussed above with respect to prior art urine receiver 30. The urine receiver 50 includes a fluid discharge tube 55 proximal the first end 57 of the pouch, the tube 55 communicating with the exterior of the pouch 51 and fluidically communicating with an interior tube or conduit 58 that extends generally along the first side 59 of the pouch. An optional support strut 62 extends generally along the second side 60 of the pouch 51, the strut 62 providing physical support for the pouch 51. Alternatively, the interior tube 54 can extend along the second side 60 of the pouch and option support strut 62 can extend along the first side 59 of the pouch. Optional spacers 63 prevent the second end 58 of the pouch from collapsing under vacuum. The strut 62 and spacers 63 are of a plastic fluid impermeable material but it is envisioned that the spacers 63 may comprise a fluid-absorbent material.

To inhibit collapse of the pouch under vacuum, the rear side of the pouch opposing the penis-receiving opening 52 may include vents 45 (shown in hidden lines in FIG. 7). The other pouches depicted in the other embodiments described herein also may include such vents. The vents may be simple perforations in the material of the pouch or they may be covered with an air-permeable material to inhibit fluid splashing.

The conduit 58 terminates in a manifold 64. The manifold 64 may be any structure that is configured to allow fluid to be drawn into the conduit 58 from multiple directions. The illustrated manifold 64 is a three-way manifold configured as a T-connector. The T-connector manifold 64 as illustrated has first and second auxiliary tubes 65, 66 that are pointed towards the first and second sides 59, 60 respectively of the pouch 51. The tubes 65, 66 terminate in curved extensions 65a, 66a, but these are optional. If the patient turns, or is rotated, with one side of the pouch 51 lower than the other, this configuration will allow suction to be applied to whichever side is lower to thereby inhibit urine build-up within the pouch 51. The support strut 62 is not a conduit but again lends structural support to the pouch 51. As shown, the penis-receiving opening 52 is disposed between the interior conduit 58 and the support strut 62.

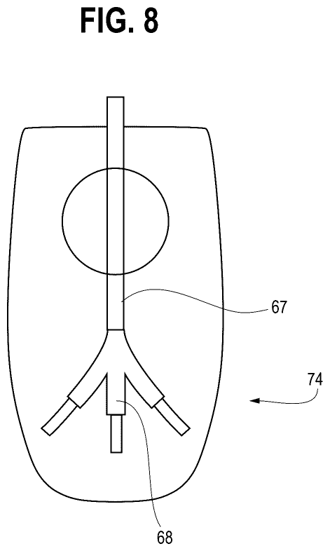

In an alternative embodiment, urine receiver 74, (as shown in FIG. 8) can include a central internal tube 67 that connects to a four-way manifold 68. Other configurations are possible and larger manifolds (with more tubing interconnections) are possible, for instance, a five-or six-way manifold. If the pouch is provided with a lining that includes spunbond-meltblown-spunbond material, this material is somewhat air-permeable and will assist in inhibiting pouch collapse.

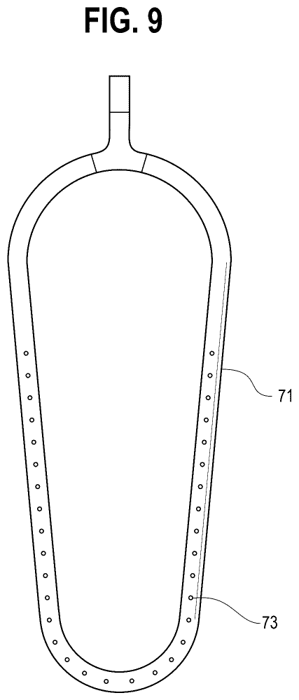

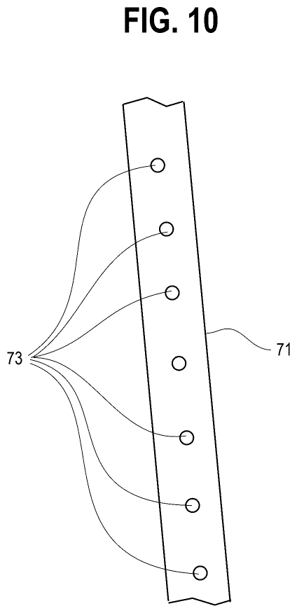

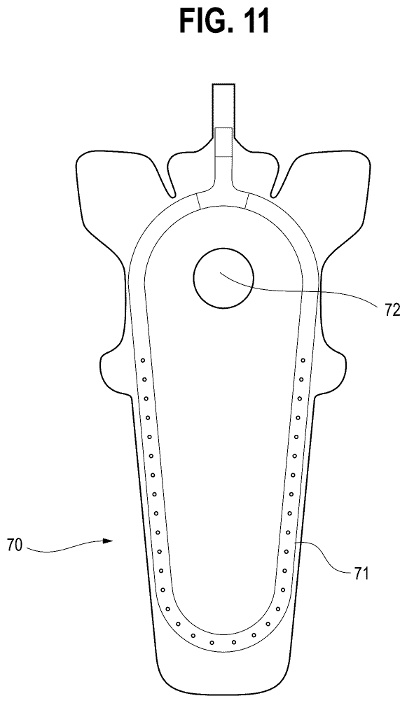

The alternative urine receiver 70 shown in FIG. 11 and whose tubing is shown in FIGS. 9-10 is configured similarly to the urine receiver 50 shown in FIGS. 7 and 8, except that the interior tubing is configured as an interior loop conduit 71 that extends generally along a first side of the pouch, across a second end of the pouch, and along a second side of the pouch. As best seen in FIG. 10, the loop conduit 71 includes a plurality of urine-permitting perforations 73. Again, the loop conduit 71 can also be used to provide additional structural support to the urine receiver 70 and is configured to allow urine to flow from each side of the urine receiver 70 even if the patient turns or is turned. The penis-receiving opening 72 is disposed within the loop conduit 71, as shown in FIG. 11.

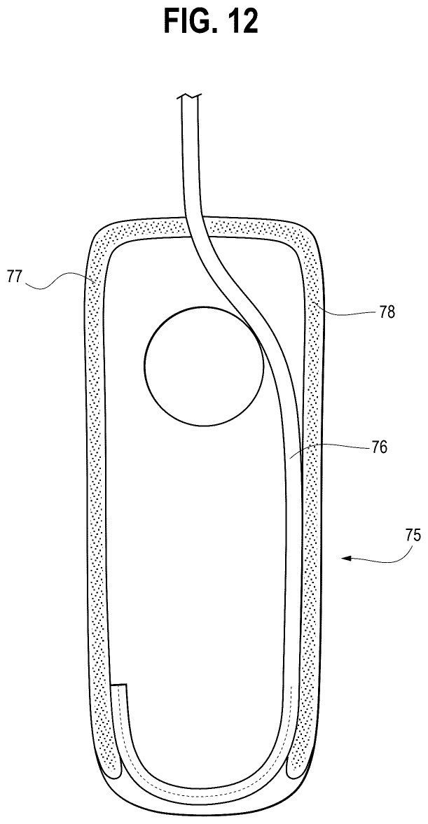

In the alternative urine receiver 75 shown in FIG. 12, a single perforated tube 76 and two support struts 77, 78 are provided. The two support struts 77, 78 and/or a perforated tube 76 may be used in configurations that include a manifold (not shown). In this and in the other illustrated embodiments, spacers (not shown in FIG. 12) may be employed.

All of the heretofore illustrated male urine receivers are used similarly. Female urine receivers, designed to be positioned and secured adjacent the vagina and female urethra, are also provided. A female urine receiver has an opening sized for placement adjacent a female urethra, which opening may be a region or field. The receiver has a fluid-impermeable back layer that is connected to a front layer via spun material or other porous material. Suitable general teachings regarding female urine receivers may be found in U.S. Pat. No. 11,813,191, incorporated by reference in its entirety. The female receivers described herein are generally useful in the system shown in FIG. 1. A caregiver may place the receiver adjacent the vagina and may connect other components of the system (tubing, suction source, urine canister) accordingly.

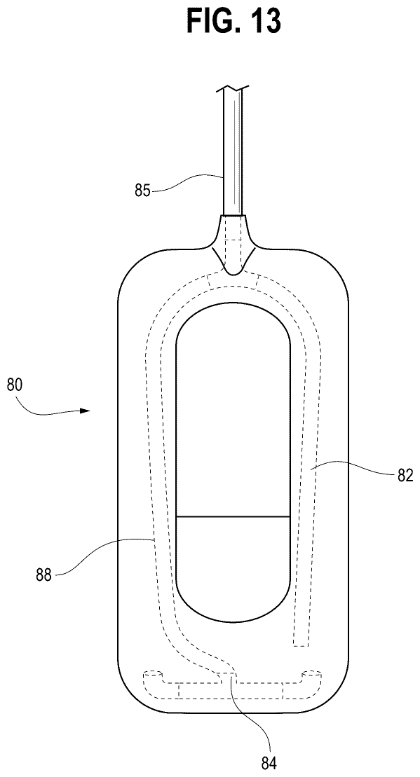

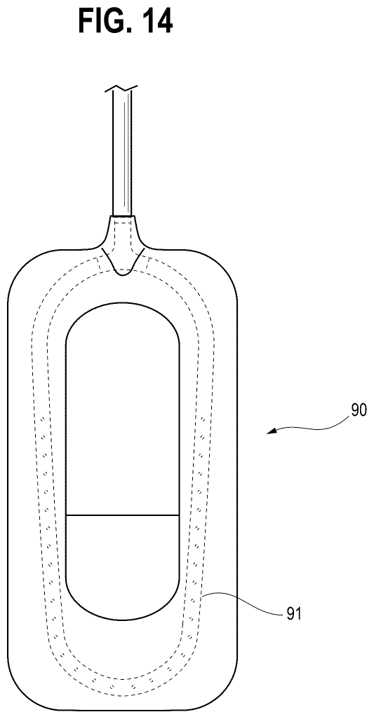

FIG. 13 generally shows a female urine receiver 80 that is generally configured as shown in U.S. Pat. No. 11,813,191 but having a discharge tube 85 that communicates with an interior conduit 88 that terminates in a manifold 84 (all interior components shown in hidden lines). In this embodiment, the manifold 84 is a three-way T-connector but a four-way or larger manifold may be employed. The conduit 88 extends along one side of the receiver 80 and an optional support strut 82 extends along the other side. Alternatively, as seen in FIG. 14, the female urine receiver 90 may be provided with a perforated loop conduit 91. In either embodiment, one or more support struts or spacers (not shown in these FIGURES) may be employed.

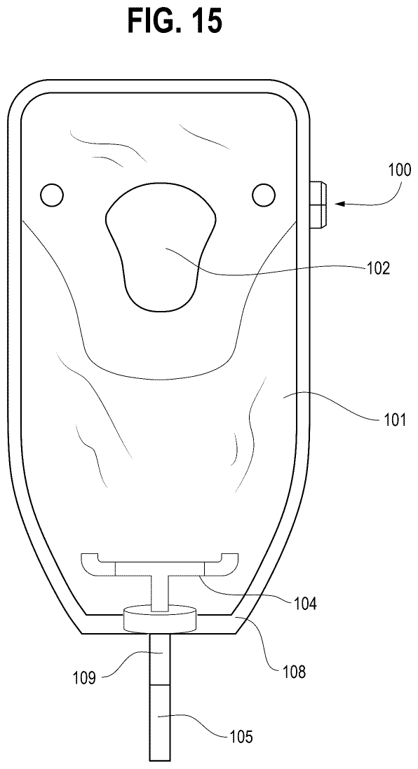

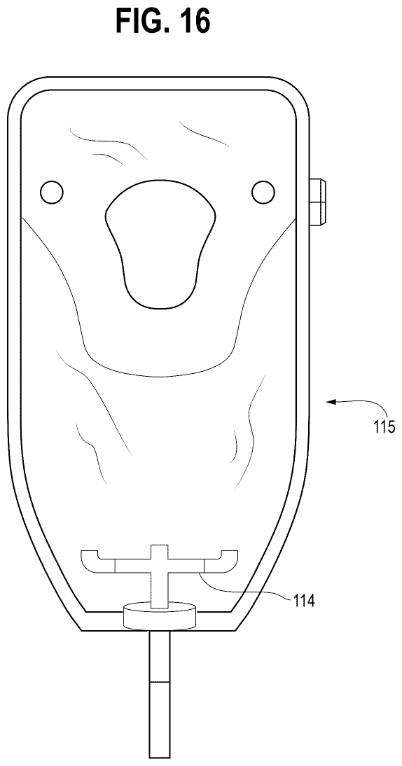

The tubing may enter the pouch near the upper portion as heretofore depicted or at the lower portion of the pouch in some embodiments. With reference to FIG. 15, the illustrated urine receiver 100 includes a pouch 101 having a penis-receiving opening 102. The lower end 108 of the pouch terminates in a fitting 109 and a discharge tube 105. The discharge tube 105 is connected to a three-way manifold 104 which is positioned to draw urine out of the pouch 101 and into the discharge tube 105. The urine receiver 115 shown in FIG. 16 is similarly configured except that it includes a four-way manifold 114.

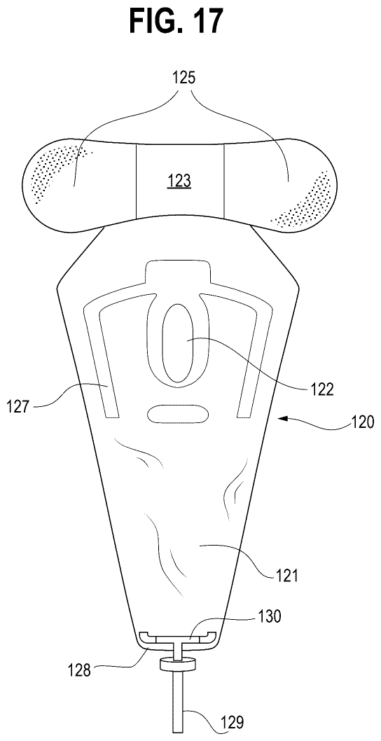

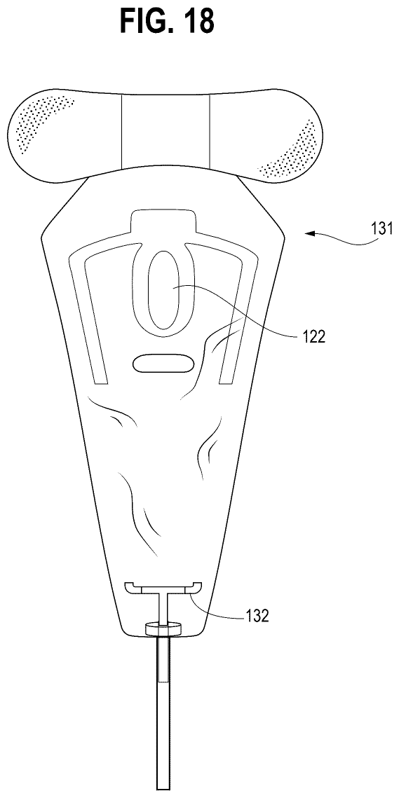

The urine receiver 120 shown in FIG. 17 includes a patient securement portion 123 with adhesive and backing strips 125. The device includes a pouch 121 with a penis-receiving opening 122 and a support 127. The lower end 128 terminates in a discharge tube 129 which is connected to a three-way manifold 130. The alternative urine receiver 131 shown in FIG. 18 is similar except that a four-way manifold 132 is provided.

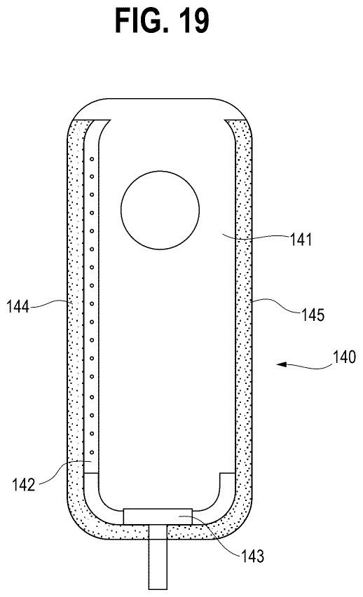

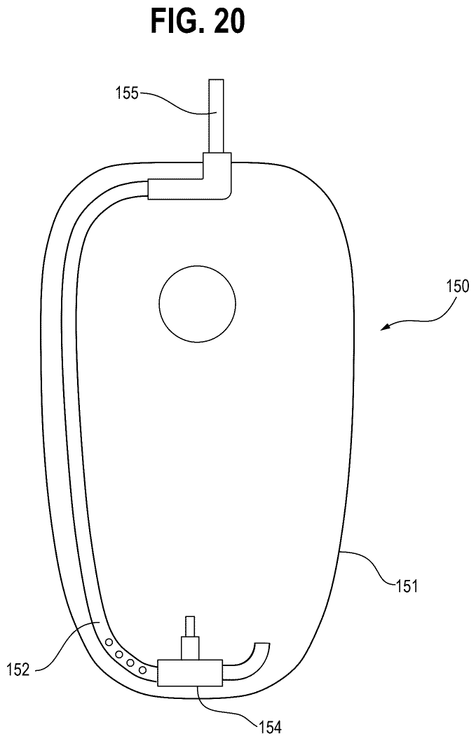

The internal tubing and components described herein can be combined in various ways. For example, as seen in FIG. 19, the urine receiver 140 has a pouch 141 with internal tubing that comprises a perforated tube 142, a three-way manifold 143 (a four-way manifold being also possible), and a pair of support struts 144, 145. Other combinations and configurations of the heretofore described components are possible. The urine receiver 150 illustrated in FIG. 20 has a pouch 151, a perforated tube 152 and a three-way manifold 154. As configured, there is no support strut but a support struct could be added. The discharge tube 155 enters the pouch 151 near the top of the device 150.

The urine receivers described herein are intended to be single-patient, disposable devices in most cases, except for the pump, which is durable and reusable. The urine receivers may be made to be reusable in some cases.

All methods described herein can be performed in any suitable order unless otherwise indicated herein or otherwise clearly contradicted by context. The use of any and all examples, or language describing an example (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

1. A urine receiver comprising
a liquid-impermeable pouch having (a) an opening sized for placement adjacent a female urethra or (b) a penis-receiving opening, said opening communicating with an interior of the pouch, the pouch having a first end and a second end and a first side and a second side;
a body-connecting region having adhesive disposed thereon for connection of the receiver to the body of a patient;
a fluid discharge tube proximal the first end of the pouch, the tube communicating with the exterior of the pouch and fluidically communicating with an interior conduit that extends generally along a first side of the pouch, the conduit terminating in a manifold.

2. The urine receiver of claim 1, including a support strut that extends generally along a second side of the pouch.

3. The urine receiver of claim 2, the opening being a penis-receiving opening and being disposed between the interior conduit and the support strut.

4. The urine receiver of claim 1, the manifold being a three-way manifold configured as a T-connector.

5. The urine receiver of claim 1, the manifold being a four-way manifold.

6. A urine receiver comprising
a liquid-impermeable pouch having (a) an opening sized for placement adjacent a female urethra or (b) a penis-receiving opening, said opening communicating with an interior of the pouch, the pouch having a first end and a second end and a first side and a second side;
a body-connecting region having adhesive disposed thereon for connection of the receiver to the body of a patient;
a fluid discharge tube proximal the first end of the pouch, the tube communicating with the exterior of the pouch and fluidically communicating with an interior loop conduit that extends generally along a first side of the pouch, across the second end of the pouch, and along the second side of the pouch, the conduit having one or more urine-permitting perforations.

7. A urine receiver comprising
a liquid-impermeable pouch having (a) an opening sized for placement adjacent a female urethra or (b) a penis-receiving opening, said opening communicating with an interior of the pouch, the pouch having a first end and a second end and a first side and a second side;
a body-connecting region having adhesive disposed thereon for connection of the receiver to the body of a patient;
a fluid discharge tube communicating with the exterior of the pouch and fluidically communicating with an interior conduit having a perforated portion, the interior conduit extending generally along a first side of the pouch; and a support strut that extends generally along a second side of the pouch.

8. A urine receiver comprising
a liquid-impermeable pouch having a first end and a second end and a first side and a second side, the pouch having a penis-receiving opening proximal the first end of the pouch, said opening communicating with an interior of the pouch;
a body-connecting region having adhesive disposed thereon for connection of the receiver to the body of a patient;
a fluid discharge tube proximal the second end of the pouch, the tube communicating with the exterior of the pouch and fluidically communicating with an interior conduit.

9. A urine receiver comprising
a liquid-impermeable pouch having a first end and a second end and a first side and a second side, the pouch having (1) a penis-receiving opening proximal the first end of the pouch, or (2) an opening sized for placement adjacent a female urethra, said opening communicating with an interior of the pouch;
a body-connecting region having adhesive disposed thereon for connection of the receiver to the body of a patient;
a fluid discharge tube communicating with the exterior of the pouch and fluidically communicating with an interior conduit, the conduit terminating in a manifold.

10. A urine receiver comprising
a liquid-impermeable pouch having (a) an opening sized for placement adjacent a female urethra or (b) a penis-receiving opening, said opening communicating with an interior of the pouch, the pouch having a first end and a second end and a first side and a second side;
a body-connecting region having adhesive disposed thereon for connection of the receiver to the body of a patient;
a fluid discharge tube communicating with the exterior of the pouch and fluidically communicating with an interior conduit having a perforated portion; and
an interior structure supporting said first side and said second side.

11. The urine receiver of claim 10, the interior structure comprising two support struts that extend generally along both sides of the pouch.

12. The urine receiver of claim 10, the opening being a penis-receiving opening and being disposed between the two support struts.

13. The urine receiver of claim 10, including one or more spacers proximal to the second end of the pouch.

14. The urine receiver of claim 10, the pouch including one or more vents.

15. The urine receiver of claim 6, the pouch including one or more spacers.

16. The urine receiver of claim 6, the pouch including one or more vents.

17. The urine receiver of claim 6, the interior conduit including multiple urine-permitting perforations.

18. A urine receiver comprising
a liquid-impermeable pouch having (a) an opening sized for placement adjacent a female urethra or (b) a penis-receiving opening, said opening communicating with an interior of the pouch, the pouch having a first end and a second end and a first side and a second side;
a body-connecting region having adhesive disposed thereon for connection of the receiver to the body of a patient;
said pouch including an interior conduit that extends generally along the first side of the pouch and that includes at least one perforation, the urine receiver permitting fluid communication from the interior of said pouch to the exterior of said pouch via said perforation; and;
a support strut that extends generally along the second side of the pouch.

19. The urine receiver of claim 18, the opening being a penis-receiving opening and being disposed between the interior conduit and the support strut.

20. The urine receiver of claim 19, said interior conduit fluidically communicating with a fluid discharge tube disposed proximal the first end of the pouch, said at least one perforation being disposed proximal the second end of the pouch.

21. The urine receiver of claim 19, including a nonwoven material disposed in the interior of said pouch.

22. The urine receiver of claim 19, said interior conduit including plural perforations.

23. The urine receiver of claim 19, said interior conduit having a portion that extends across at least a portion of the second end of the pouch.

24. The urine receiver of claim 23, said interior conduit including an open end, said open end proximal said second side of said pouch and said at least one perforation proximal said first side of said pouch.
